(19) United States
(12) Patent Application Publication
(10) Pub. No.: US 2026/0048209 A1
(43) Pub. Date: Feb. 19, 2026

(54) DRY-POWDER INHALER

(71) Applicant: APTAR FRANCE SAS, LE NEUBOURG (FR)

(72) Inventor: Franck POULLAIN, LA HAYE MALHERBE (FR)

(73) Assignee: APTAR FRANCE SAS, LE NEUBOURG (FR)

(21) Appl. No.: 19/103,943

(22) PCT Filed: Aug. 16, 2023
(86) PCT No.: PCT/FR2023/051275

§ 371 Date: Feb. 14, 2025

(30) Foreign Application Priority Data
Aug. 18, 2022 (FR) FR2208371

(51) Int. Cl. A61M 15/00

(52) U.S. Cl. CPC A61M 15/0008; A61M 15/0021; A61M 15/0086; A61M 2202/064; A61M 2206/16

(57) ABSTRACT

A dry-powder inhaler having a body and a mouthpiece provided with a dispensing orifice through which the user inhales, a chamber, a reservoir containing a dose of dry powder to be inhaled and having a lateral outlet opening. The reservoir is disposed in the chamber, the diameter of the reservoir being less than the diameter of the chamber, so that, in the loaded position, the reservoir can move in the chamber. The body has tangential channels adapted to bring a flow of inhalation air tangentially into the chamber, which, during inhalation, generates an orbital movement of the reservoir in the chamber, in the loaded position. The reservoir has an external profile defining an inscribed volume forming an external volume of the reservoir that is greater than the internal volume of the reservoir that contains the powder.

The present invention relates to a dry-powder inhaler.

Dry-powder inhalers are well known in the prior art. Various types exist.

A first type of multidose inhaler contains a reservoir receiving multiple doses of powder, the inhaler being provided with dosage means, making it possible, on each actuation, to separate out one dose of this powder from the reservoir in order to bring it into an expulsion duct so that it can be dispensed to the user. Such devices require complicated and therefore expensive dosage means, and dosage accuracy and reproducibility are not guaranteed. In addition, there are risks of contamination of the powder disposed in the reservoir as the inhaler is being used.

Another type of multidose inhaler consists of providing several individual reservoirs each containing one dose of powder, then opening one of those reservoirs each time the inhaler is actuated. That implementation ensures that the powder is better sealed, since each dose of the powder is only exposed to the atmosphere at the moment when it is expelled. In order to produce such sets of individual reservoirs, different variations have already been proposed, such as blister strips or disks. With regard to opening the individual reservoirs, peeling off or unsticking the closure layer of the blisters has been proposed. That suffers from the disadvantage of difficulty in controlling the forces to be applied in order to guarantee full opening, without running the risk of opening the next reservoir, particularly if the opening means need to be actuated by inhalation. Another solution is to pierce the closure membrane of a blister on each actuation, which requires complicated and therefore expensive piercing means.

In addition, the multidose inhalers described above suffer from the problem of not guaranteeing optimal dispensing effectiveness, with a significant portion of the dose effectively penetrating into the user's lungs in order to have a beneficial therapeutic effect.

In order to make devices less complicated and thus less expensive, single-dose inhalers have been proposed which comprise a single individual reservoir, such as a blister or a capsule, which has to be loaded into the inhaler just before it is used. The advantage of such single-dose devices is that it is not necessary to store all of the doses inside the device, so that the dimensions thereof can be reduced. In contrast, it is more complicated for the user to use, since they are obliged to load a blister or a capsule into the inhaler before each use. Furthermore, other disadvantages specific to these single-dose inhalers exist. Thus, a blister or capsule does not allow large doses, typically greater than 100 mg, to be dispensed. In addition, in particular as regard capsules or blisters to be pierced, opening requires the use of complicated piercing means, and also risks getting residues of pierced membrane in the powder which is dispensed. Furthermore, the emptying performance of the reservoir is not always optimal, since a portion of the powder could remain inside the reservoir during inhalation.

In an attempt to overcome these disadvantages, document WO 98/26828 proposes a single-dose inhaler comprising a cylindrical reservoir provided with a lateral opening, disposed in a cylindrical chamber with a larger diameter, in a manner such that during inhalation, the reservoir is displaced in the chamber in accordance with an orbital movement, which causes the powder to be expelled. This implementation effectively makes it possible to improve the efficiency of dispensing, with substantially complete emptying of the reservoir and a larger portion of the dose which penetrates effectively into the lungs. Nevertheless, a disadvantage of this device is that the size of the reservoir must be well adapted to that of the chamber to ensure good orbital movement. However, unless the inhaler is modified, which would be very expensive, this does not make it possible to easily vary the dose of powder, whereas it may be desirable to be able to use the same inhaler with reservoirs containing different dosages.

Documents WO02085281 A1 WO03075988A1, WO2009121020A1, WO2013008037A1 and WO2008001132A1 describe other devices of the state of the art.

An object of the present invention is to provide a powder inhaler that does not have the above-mentioned drawbacks.

In particular, an object of the present invention is to provide an inhaler of this type that improves dispensing efficiency, by allowing almost all of the powder contained in the reservoir to be dispensed and by increasing the portion of the dose that will reach the user's lungs.

The present invention also aims to provide an inhaler of this type which optimize the displacement of the reservoir in the chamber during actuation.

Another objective of the present invention is to provide an inhaler of this type which reduces the complexity of use for the user and which guarantees better safety in use.

Another objective of the present invention is to provide an inhaler of this type which is simple and inexpensive to manufacture and to assemble.

The present invention thus provides a dry-powder inhaler comprising:
a body and a mouthpiece provided with a dispensing orifice through which the user inhales,
a chamber,
a reservoir containing a dose of dry powder to be inhaled and comprising a lateral outlet opening, said reservoir being disposed in said chamber, the diameter of said reservoir being less than the diameter of said chamber, so that, in the loaded position, said reservoir can move in said chamber, said body comprising tangential channels adapted to bring a flow of inhalation air tangentially into said chamber, which, during inhalation, generates an orbital movement of said reservoir in said chamber, in the loaded position,

said reservoir comprising an external profile defining an inscribed volume forming an external volume of the reservoir greater than the internal volume of the reservoir that contains the powder.

Advantageously, said reservoir is formed by an upper reservoir portion and a lower reservoir portion, which are assembled after filling with the dose of powder.

Advantageously, each reservoir portion has its respective profile.

Advantageously, said lower reservoir portion comprises said lateral outlet opening.

Advantageously, said external profile comprises a plurality of ribs extending radially on an external wall of said reservoir.

Advantageously, said mouthpiece comprises a grid upstream from said dispensing orifice.

Advantageously, said chamber is disposed between an inhalation air inlet and said mouthpiece.

Advantageously, said reservoir contains a dose of powder greater than 50 mg, in particular greater than 100 mg.

Advantageously, the volume occupied by said reservoir in said chamber in the loaded position is greater than 50% of the volume of said chamber.

Advantageously, the radial diameter of said reservoir is greater than its axial height.

Advantageously, said chamber is disposed in a loading member mounted so as to be movable, in particular pivoting, on said body between an non-loaded position, in which a reservoir can be disposed in said chamber, and a loaded position, in which said chamber and said reservoir are disposed inside said body of the inhaler.

According to an advantageous variant, the chamber is disposed in said body, said inhaler comprising an actuating member axially displaceable with respect to said body in order to displace said reservoir from a non-loaded position towards a loaded position.

Advantageously, said actuating member comprises at least one oblique groove receiving at least one respective lug formed on said body or on an element which is secured to said body, in a manner such that during its axial displacement, said actuating member implements a rotation in said body.

These features and advantages and others of the present invention will appear more clearly during the following detailed description, made in reference to the accompanying drawings, given as non-limiting examples, and in which

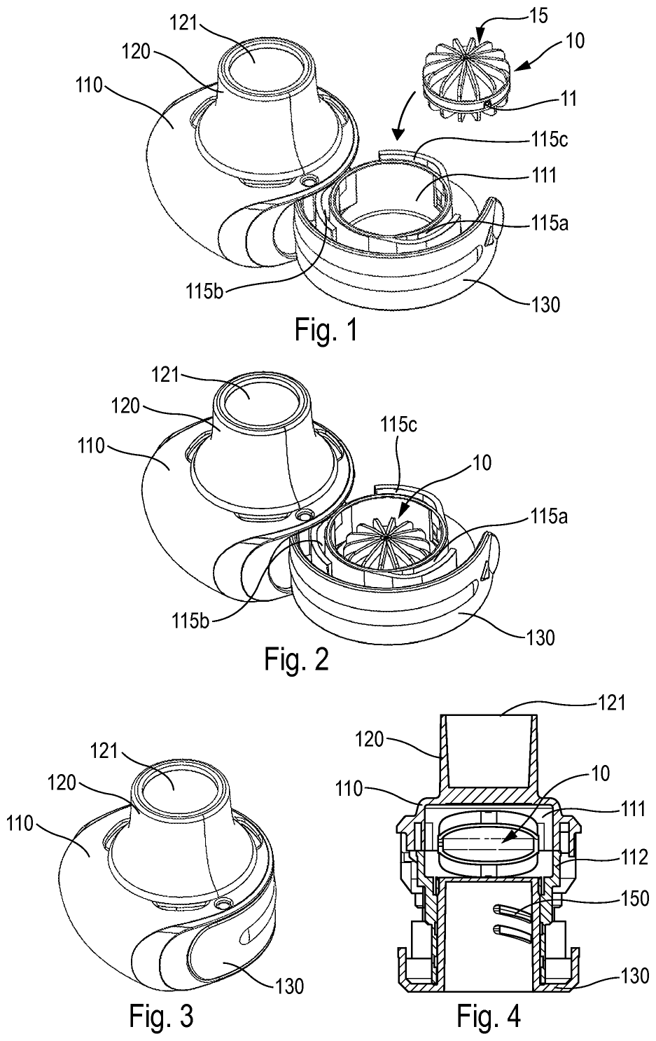

FIG. 1 is a diagrammatic perspective view of an inhaler according to a first advantageous embodiment, before inserting the reservoir in the inhaler,

FIG. 2 is a view similar to the view in FIG. 1, after assembling the reservoir, but in the non-loaded position.

FIG. 3 is a view similar to that of FIG. 2, in the loaded position,

FIG. 4 is a diagrammatic cross-sectional view of an inhaler in accordance with a second advantageous embodiment, in the loaded position,

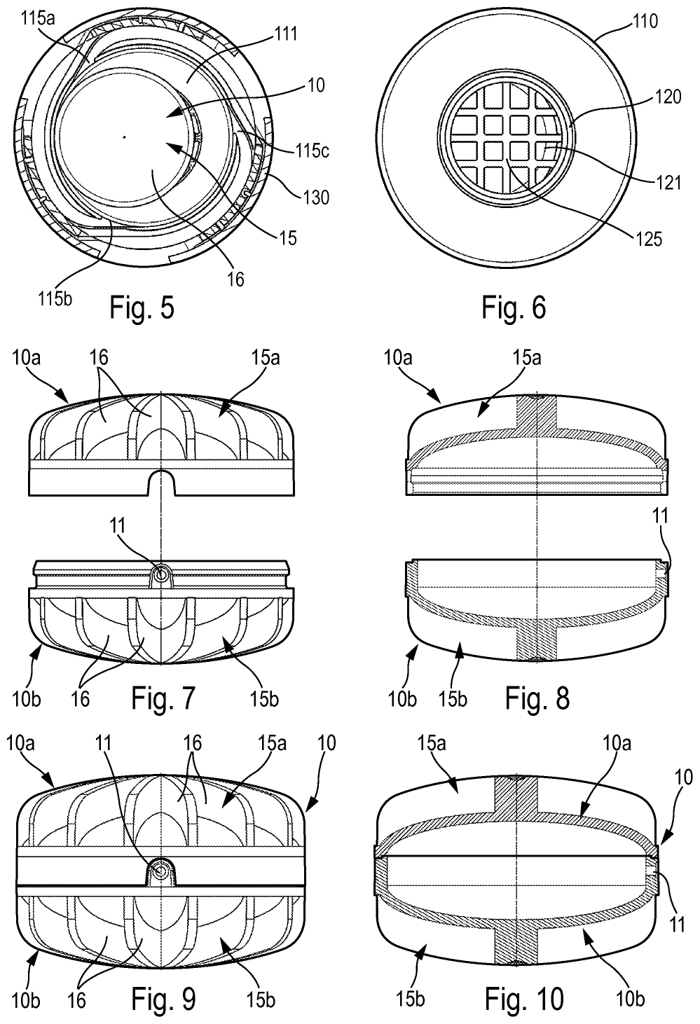

FIG. 5 is a diagrammatic view from below of the reservoir in the chamber, in the loaded position,

FIG. 6 is a diagrammatic view from above of the mouthpiece.

FIG. 7 is a diagrammatic perspective view from below of the reservoir, before closing,

FIG. 8 is a diagrammatic section view of the device of FIG. 7,

FIG. 9 is a diagrammatic perspective view of the reservoir, after closing, and

FIG. 10 is a diagrammatic section view of device of FIG. 9.

In the description below, the terms “upper”, “lower” and “lateral” are with respect to the upright position of the device shown in FIG. 4. The terms “axial” and “radial” are relative to the longitudinal central axis A of the device.

FIGS. 1 to 3 describe a first advantageous embodiment.

In this first embodiment, the inhaler comprises a body 110 and a mouthpiece 120 provided with a dispensing orifice 121 through which the user inhales. A loading member 130 containing a chamber 111 is mounted to move, advantageously pivot, on the body 110, between a non-loaded position and a loaded position. In the non-loaded position, a reservoir 10 may be disposed in the chamber 111, and in the loaded position, shown in FIG. 3, the chamber 111 and the reservoir 10 are inside the body 110 of the inhaler.

Advantageously, as can be seen in FIG. 6, the mouthpiece 120 includes a grid 125 upstream from the dispensing orifice 121.

Preferably, the chamber 111 is disposed between an inhalation air inlet and said mouthpiece 120, in a manner such that when the user inhales through the mouthpiece 120, the air flow will pass through said chamber 111.

The inhaler comprises a reservoir 10 containing a dose of dry powder to be inhaled and comprising a lateral outlet opening 11.

The diameter of the reservoir 10 is less than the diameter of said chamber 111, in a manner such that in the loaded position, the reservoir 10 can be displaced in the chamber 111. Tangential channels 115a, 115b, 115c bring said flow of inhalation air tangentially into the chamber 111, thereby causing the reservoir 10 to move in the chamber 111 in an orbital manner, the reservoir 10 rotating on itself while rotating about the periphery of the chamber 111 along the side wall thereof. This movement will allow the powder contained in the reservoir 10 to be expelled, which is entrained by the flow of inhalation air towards the mouthpiece and the dispensing orifice.

Advantageously, the reservoir 10 may contain a dose of powder greater than 50 mg, in particular greater than 100 mg. Optionally, very large doses may be envisaged, in particular greater than 500 mg.

Advantageously, the volume occupied by the reservoir 10 in the chamber 111 in the loaded position is greater than 50% of the volume of the chamber 111.

Advantageously, the radial diameter of the reservoir 10 is greater than its axial height.

Advantageously, the reservoir 10 is formed by an upper reservoir portion 10a and a lower reservoir portion 10b, which are assembled after filling with the dose of powder. Advantageously, it is the lower reservoir portion 10b which comprises the lateral outlet opening 11. Advantageously, the lateral outlet opening 11 is disposed in an axially centered manner.

Advantageously, the reservoir 10 is symmetrical, such that it can be inserted into the chamber 111 equally well in both directions.

FIG. 4 describes a second advantageous embodiment.

In this second embodiment, the inhaler comprises a body 110 containing a chamber 111 and a mouthpiece 120 provided with a dispensing orifice 121 through which the user inhales.

Advantageously, the body 110 is formed by two body portions, an upper body portion comprising the mouthpiece 120 and a lower body portion comprising a cylindrical sleeve 112, the chamber 111 being defined by these two body portions fixed to one another.

The inhaler also comprises an actuating member 130 for displacing said reservoir 10 from its non-loaded position towards its loaded position. This actuating member 130 is axially displaceable with respect to the body 110 in order to push the reservoir 10 from its non-loaded position towards its loaded position.

Advantageously, the axial displacement of the actuating member 130 is produced by a rotation of the actuating member 130 in the body 110, for example by means of one or more lugs of the body 110 disposed in one or more oblique grooves 150 of the actuating member 130.

According to the invention, the reservoir 10 comprises an external profile 15 defining an external volume of the reservoir that is greater than the internal volume of the reservoir that contains the powder. This implementation makes it possible to optimize the external dimension of the reservoir 10 relative to the dimension of the chamber 111, in order to ensure good orbital movement of the reservoir 10 during actuation. At the same time, the internal volume of the reservoir can be modified, while always keeping the same external volume, which makes it possible to use reservoirs with different powder dosages in the same inhaler.

Advantageously, each reservoir portion 10a, 10b has its respective profile 15a, 15b.

Advantageously, the external profile 15 comprises a plurality of ribs 16 extending radially on an external wall of said reservoir 10. The number of ribs can be any number, as long as they define an inscribed volume forming the external volume of the reservoir.

The device of the invention is simple and effective. It is made up of a small number of parts and is thus inexpensive to manufacture and to assemble, and is reliable in use. It allows optimal dispensing of the powder due to the orbital movement of the reservoir during actuation, while guaranteeing the integrity of the powder until it is used.

It should be noted that the inhaler is refillable, by removing the empty reservoir and replace it with a full reservoir.

Various modifications can also be envisaged by a person skilled in the art, without going beyond the ambit of the present invention, as defined by the accompanying claims.

1. A dry-powder inhaler comprising:
a body and a mouthpiece provided with a dispensing orifice through which the user inhales,
a chamber,
a reservoir containing a dose of dry powder to be inhaled and comprising a lateral outlet opening said reservoir being disposed in said chamber the diameter of said reservoir being less than the diameter of said chamber, so that, in the loaded position, said reservoir can move in said chamber said body comprising tangential channels adapted to bring a flow of inhalation air tangentially into said chamber which, during inhalation, generates an orbital movement of said reservoir in said chamber in the loaded position,
characterized in that said reservoir comprises an external profile 15 defining an inscribed volume forming an external volume of the reservoir that is greater than the internal volume of the reservoir that contains the powder.

2. The inhaler according to claim 1, wherein said reservoir is formed by an upper reservoir portion and a lower reservoir portion which are assembled after filling with the dose of powder.

3. The inhaler according to claim 2, wherein each reservoir portion has its respective profile.

4. The inhaler according to claim 2, wherein said lower reservoir portion comprises said lateral outlet opening

5. The inhaler as claimed in claim 1, wherein said external profile comprises a plurality of ribs extending radially on an outer wall of said reservoir.

6. The inhaler as claimed in claim 1, wherein said mouthpiece comprises a grid upstream from said dispensing orifice

7. The inhaler as claimed in claim 1, wherein said chamber is disposed between an inhalation air inlet and said mouthpiece

8. The inhaler as claimed in claim 1, wherein said reservoir contains a dose of powder greater than 50 mg, in particular greater than 100 mg.

9. The inhaler as claimed in claim 1, wherein the volume occupied by said reservoir in said chamber in the loaded position is greater than 50% of the volume of said chamber.

10. The inhaler as claimed in claim 1, wherein the radial diameter of said reservoir is greater than its axial height.

11. The inhaler as claimed in claim 1, wherein said chamber is disposed in a loading member mounted so as to be movable, in particular pivoting, on said body 110 between a non-loaded position, in which a reservoir can be disposed in said chamber and a loaded position, in which said chamber and said reservoir are disposed inside said body of the inhaler.

12. The inhaler as claimed in claim 1, wherein said chamber is disposed in said body, said inhaler comprising an actuating member axially movable with respect to said body in order to move said reservoir from a non-loaded position towards a loaded position.

13. The inhaler according to claim 12, wherein said actuating member comprises at least one oblique groove receiving at least one respective lug formed on said body or on an element which is secured to said body, in a manner such that during its axial displacement, said actuating member implements a rotation in said body
